(19) United States
(12) Patent Application Publication
(10) Pub. No.: US 2012/0209058 A1
(43) Pub. Date: Aug. 16, 2012

(54) Therapeutic pillow

(76) Inventor: Katayoun Soroush Arasi, (US)

(21) Appl. No.: 13/025,119

(22) Filed: Feb. 10, 2011

(51) Int. Cl. A61M 21/02; A61Q 19/10; C11D 3/382; A61Q 13/00; A61K 36/752; A61K 8/97

(52) U.S. Cl. 600/27; 424/736; 512/1; 510/138; 510/159

(57) ABSTRACT

A device for providing aromatherapy is disclosed herein. The aromatherapy device comprises a textile article and aromatic materials disposed upon or within the textile article. The aromatic materials are the source for generating or evaporating aromatic scent to function on human mind or body.

### BACKGROUND OF THE INVENTION

This invention relates to a device and method for applying aromatherapy.

Aromatherapy is a form of therapy using volatile aromatic materials to improve a person's mood, health, cognitive function or mind. The therapy generates aromatic scent, which stimulates the brain, especially the limbic system through the olfactory system, resulting in feels of relaxation, clarity of mind, healing effects, etc.

Conventional applications of aromatherapy involve aerial diffusion process to disperse the aromatic substance for inhalation. The diffusing process requires apparatuses or devices such as those disclosed in U.S. Pat. No. 6,053,483, entitled “Aroma Diffuser”; and U.S. Patent Application Number 2008/0299014, entitled “Aroma Diffuser for Vehicles.”

Aromatherapy has eminent effect to alleviate and relieve symptoms or discomfort caused by long time travel, including motion sickness, nausea, headache, dry and irritated skin, flight phobia, cluster phobia, restlessness, ear ache, etc. However, given the time and space limitations during travel, using the conventional techniques to perform aromatherapy is extremely inconvenient, if not impossible. Thus, long time need calls for innovative techniques to apply aromatherapy, which can be conveniently implemented during either travel or daily use.

### SUMMARY OF THE INVENTION

Given the above deficiencies of conventional techniques to implement aromatherapy, the present invention seeks to address these problems by adding therapeutic substances to travel accessories, such as travel pillows, eye masks, ear covers, blankets, throws, bags, body sprays, after shave cream and lotion, for example. This invention's application, however, is not limited to travel accessories only, but can also be extended to home accessories, such as comforters, pillows, mattress pads, cushions, shams, towels and anything suitable for containing aromatic substances.

An innovative device for providing aromatherapy is disclosed herein. The aromatherapy device includes a textile article and aromatic materials disposed upon or within the textile article. The aromatic materials are used for generating aromatic scent to function on human mind or body.

In some embodiments, the textile article is a travel pillow, preferably including a U-shaped cushion for neck resting. The textile article can also be a regular pillow for daily use, an eye mask, an ear cover, a blanket, a throw, a bedding sheet, a comforter, a cover, a bag, a purse, garment, a towel, etc.

Advantageously, the textile article according to the present invention includes a hollow structure for containing the aromatic material. In addition, the aromatic material can be in the form of particles and interwoven with the fabrics of the textile article.

The aromatic material includes one or more of the following substances: marjoram, lavender, roman chamomile, rose, fennel, cypress, cedar wood, sandalwood, eucalyptus, peppermint, geranium; vanilla, grapefruit, mandarin, orange, tangerine, and lemon.

A solution for providing aromatherapy is also disclosed herein. The aromatherapy solution comprises a fluid, and at least one aromatic material resolved in the fluid for generating scent. The suitable fluid type can be body sprays, soap, hand wash, body wash, bath gel, shower gel, perfume, cologne, after shave cream and lotion.

These and other features and advantages of this invention will become further apparent from the detailed description and accompanying figures that follow. In the figures and description, numerals indicate the various features of the invention, like numerals referring to like features throughout both the drawings and the description.

### BRIEF DESCRIPTION OF THE DRAWINGS

FIG. 1 illustrates an exemplary embodiment of an aromatic pillow according to the present invention.

FIG. 2 is a cross sectional view of the aromatic pillow illustrated in FIG. 1.

FIG. 3 illustrates an aromatic eye mask according to another embodiment of the present invention.

FIG. 4 provides a cross sectional view of the aromatic eye mask illustrated in FIG. 3.

FIG. 5 is an aromatic blanket 500 according to one embodiment of the present invention.

### DETAILED DESCRIPTION OF THE PREFERRED EMBODIMENT(S)

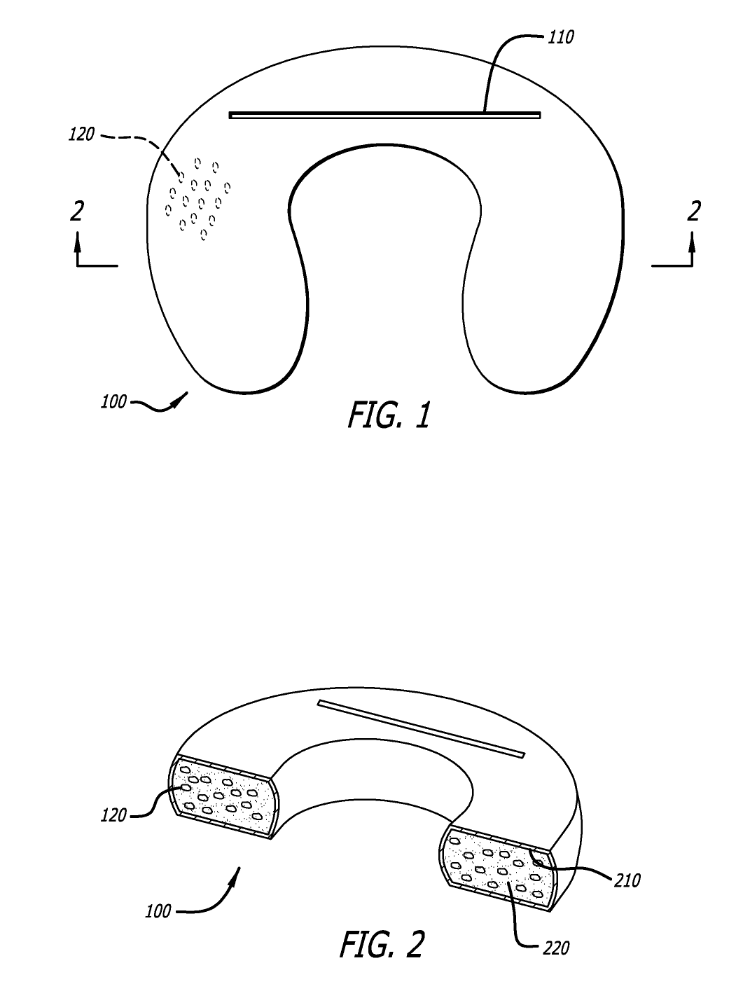

FIG. 1 illustrates an exemplary embodiment of aromatic pillow 100 according to the present invention. Although the aromatic pillow 100 is in the form of travel pillow, but can also be embodied in regular pillows, shams, cushions, mattress pads, or the like. The aromatic pillow 100 is designed for appropriate neck and support while traveling. It is preferably U-shaped so as to wrap around and cradle a user's neck to provide comfort and reduce neck stress.

FIG. 2 is a cross sectional view of the aromatic pillow 100 of FIG. 1. The aromatic pillow 100 comprises a hollow structure 210 which contains a filler 220 therein. Traditionally the filler 220 is made from foam, synthetic fibers, cottons, wools, feathers, or down, for example. The filler 220 can be inserted into and/or taken out from the aromatic pillow 100 through a long opening 110 that is normally disposed in the back of aromatic pillow 100. Advantageously, the long opening 110 is closed and opened with fasteners such as zippers, hook and loop strips, buttons, etc.

The aromatic pillow 100 also comprises aromatic substances 120 made of herbs for generating fragrance or aroma to achieve therapeutic effects. Thousands of herbs have been used in aromatherapy throughout the history. By way of example, herbs commonly used include marjoram, lavender, roman chamomile, rose, fennel, cypress, cedar wood, sandalwood, eucalyptus, peppermint, geranium, grapefruit, mandarin, orange, tangerine, lemon, etc. Depending on the purposes of aromatic treatment, any one of these herbs can be utilized alone or blended with one or more of other materials for evaporating therapeutic fragrance.

Different aromatic herbs and combinations thereof are known to have different effects or benefits to humans. For instance, some herbal chemicals generate scent which is clinically proven to reduce stress or discomfort level, such as lavender or sandalwood. Relevant experiments show that the combination of roman chamomile, spike lavender, geranium, and vanilla in the ratio of 5:3:3:1 relieves anxiety and panic attacks. Marjoram, lavender, roman chamomile and mandarin mixed according to the ratio of 2:2:4:3 aids to relax and please the mind, thereby inducing peaceful sleep.

For users engaged in long time travel or long work hours, aromatic pillow 100 with the aforementioned combinations of herbs provides a handy means for them to conveniently receive the aromatic treatment, which, traditionally would require additional apparatuses such as diffusers or infusers to conduct.

Preferably, the aromatic substances 120 are disposed in the hollow structure 210 of the aromatic pillow 100, either alone or along with traditional fillers such as cottons, foam, synthetic fillers, feathers, down, wools, etc. In some embodiments, the aromatic substances 120 are refined into particles, which can be integrally formed as part of the fabrics or interwoven into the textile of the aromatic pillow 100.

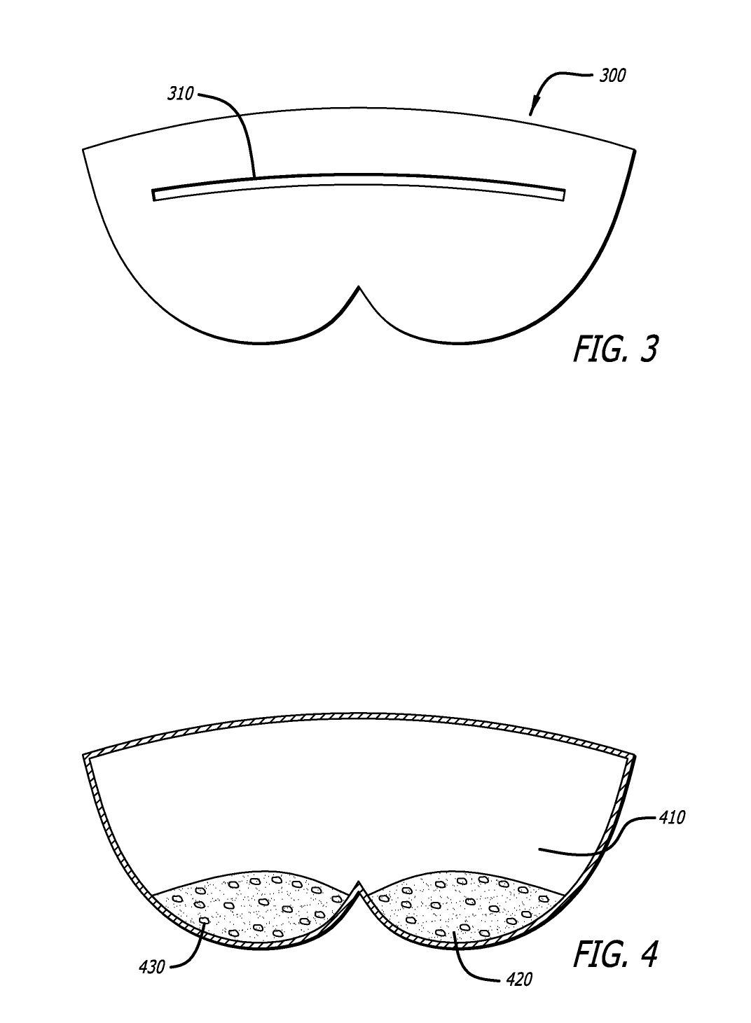

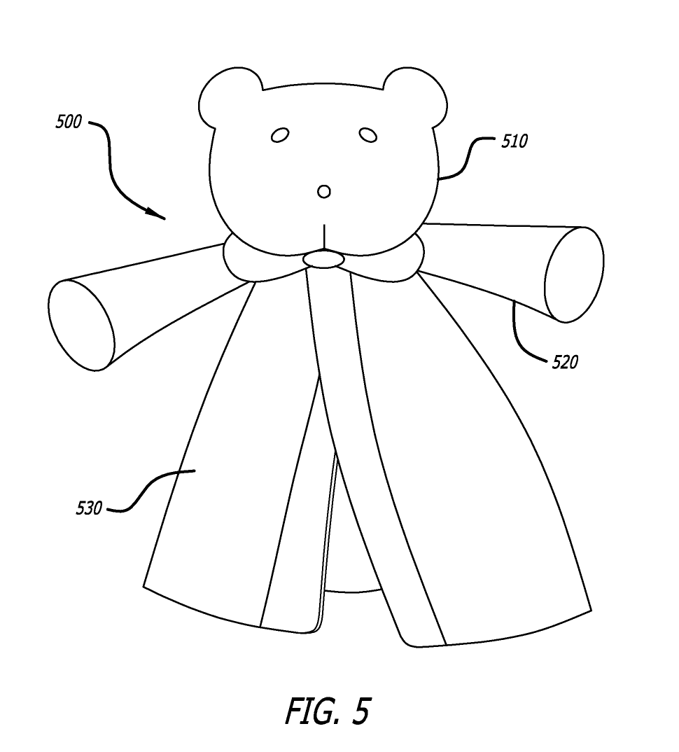

FIG. 3 illustrates another embodiment according to the present invention. Aromatic eye mask 300 is an eye mask or a sleep mask, used for aiding users to fall asleep or enhancing sleep quality. As shown in FIG. 4, which provides a cross sectional view of the aromatic eye mask 300, the eye mask 300 has a hollow structure 410 therein. Filler 420 such as synthetics, gel, fluid or the like can be used to fill in the hollow structure 410. In some embodiments, the filler 420 is inserted into and/or taken out from a long opening 310, that is normally disposed in the back of the aromatic eye mask 300. The long opening 310 is closed and opened advantageously with fasteners such as zippers, hook and loop strips, buttons, etc.

As with the aromatic pillow 100, the aromatic substances 420 can be disposed in the hollow structure 410, either alone or along with conventional filler materials. In various embodiments, they are formed integrally with the fabrics and textiles of the eye mask 100 that generally constitute the cover thereof.

In addition to pillows and eye masks, the present invention can be embodied in a diversity of textile articles, including towels, throws, bags, purses, mattress pads, cushions, shams, towels, etc.

FIG. 5 is an aromatic blanket 500 according to one embodiment of the present invention. Head portion 510 of the aromatic blanket 500 has a hollow structure (not shown) therein to contain fillers (not shown), such as conventional materials or aromatic herb substances. Arm portion 520 of the aromatic blanket 500 may also comprise a hollow structure to contain fillers of conventional materials or herbs. Advantageously, blanket portion 530 is made of textiles having aromatic substance particles integrally formed therewith.

As set forth above, conventional materials used for fillers comprise cottons and synthetic fibers, for example. Herb substances comprise lavender buds, sandalwood, roman chamomile, rose, fennel, cypress, cedar wood, sandalwood, eucalyptus, peppermint, geranium, grapefruit, mandarin, orange, tangerine, lemon, etc.

In various embodiments of the present invention, the herb substances used for aromatherapy are resolved in fluid for application on human skin. The fluid type that is suitable for such use includes skin care products such as body sprays, soap, hand or body wash, bath or shower gel, perfume, cologne, after shave cream, lotion, etc. Herbs such as rose and lavender, for example, are often utilized in this aromatherapy application to sooth and moisturize skin.

Having now described the invention in accordance with the requirements of the patent statutes, those skilled in this art will understand how to make changes and modifications in the present invention to meet their specific requirements or conditions. Such changes and modifications may be made without departing from the scope and spirit of the invention as set forth in the following claims.

What is claimed is:

1. A device for providing aromatherapy, comprising:
a textile article; and
at least one aromatic material disposed upon or within the textile article for generating scent.

2. The device of claim 1, wherein the textile article includes a cushion for neck resting.

3. The device of claim 2, wherein the cushion is U-shaped for wrapping around a user's neck.

4. The device of claim 1, wherein the textile article includes one of the following:
a pillow;
an eye mask;
an ear cover;
a blanket;
a throw;
a bedding sheet;
a comforter;
a cover;
a bag;
a purse;
garment; and
a towel.

5. The device of claim 1, wherein the textile article includes a hollow structure for containing the aromatic material.

6. The device of claim 1, wherein the aromatic material is in the form of particles and interwoven with the fabrics of the textile article.

7. The device of claim 1, wherein the aromatic material includes one or more of the following substances:
marjoram;
lavender;
roman chamomile;
rose;
fennel;
cypress;
cedar wood;
sandalwood;
eucalyptus;
peppermint;
geranium;
vanilla;
grapefruit;
mandarin;
orange;
tangerine; and
lemon.

8. A solution for providing aromatherapy, comprising:
a fluid; and
at least one aromatic material resolved in the fluid for generating scent.

9. The solution of claim 8, wherein the fluid includes one of the following:
body spray;
soap;
hand wash;
body wash;
bath gel;
shower gel;
perfume;
cologne;
after shave cream; and
lotion.

10. The solution of claim 8, wherein the aromatic material includes one or more of the following substances:
marjoram;
lavender;
roman chamomile;
rose;
fennel;
cypress;
cedar wood;
sandalwood;
eucalyptus;
peppermint;
geranium;
vanilla;
grapefruit;
mandarin;
orange;
tangerine; and
lemon.

11. A method for providing aromatherapy, comprising:
providing a textile article; and
disposing at least one aromatic material upon or within the textile article for generating scent.

12. The method of claim 11, wherein the textile article includes a cushion for neck resting.

13. The method of claim 12, wherein the cushion is U-shaped for wrapping around a user's neck.

14. The method of claim 11, wherein the textile article includes one of the following:
a pillow;
an eye mask;
an ear cover;
a blanket;
a throw;
a bedding sheet;
a comforter;
a cover;
a bag;
a purse;
garment; and
a towel.

15. The method of claim 11, wherein the textile article includes a hollow structure for containing the aromatic material.

16. The device of claim 11, wherein the aromatic material is in the form of particles and interwoven with the fabrics of the textile article.

17. The method of claim 11, wherein the aromatic material includes one or more of the following substances:
marjoram;
lavender;
roman chamomile;
rose;
fennel;
cypress;
cedar wood;
sandalwood;
eucalyptus;
peppermint;
geranium;
vanilla;
grapefruit;
mandarin;
orange;
tangerine; and
lemon.

18. A method for providing aromatherapy, comprising:
providing a fluid; and
resolving at least one aromatic material in the fluid for generating scent.

19. The method of claim 18, wherein the fluid includes one of the following:
body spray;
soap;
hand wash;
body wash;
bath gel;
shower gel;
perfume;
cologne;
after shave cream; and
lotion.

20. The method of claim 18, wherein the aromatic material includes one or more of the following substances:
marjoram;
lavender;
roman chamomile;
rose;
fennel;
cypress;
cedar wood;
sandalwood;
eucalyptus;
peppermint;
geranium;
vanilla;
grapefruit;
mandarin;
orange;
tangerine; and
lemon.
